(19) United States
(12) Patent Application Publication
(10) Pub. No.: US 2009/0306221 A1
(43) Pub. Date: Dec. 10, 2009

(54) Use for Cannabinoid

(75) Inventors: Geoffrey Guy, Wiltshire (GB); Roger Pertwee, Wiltshire (GB); Adele Thomas, Aberdeen (GB)

Correspondence Address: WOLF GREENFIELD & SACKS, P.C., 600 ATLANTIC AVENUE, BOSTON, MA, 02210-2206, US

(73) Assignee: GW PHARMA LIMITED, SALISBURY WILTSHIRE (GB)

(21) Appl. No.: 12/227,886

(22) PCT Filed: May 31, 2007
(86) PCT No.: PCT/GB2007/002008

§ 371 Date: Jun. 22, 2009

(30) Foreign Application Priority Data
Jun. 1, 2006 (GB) 0610828.6

(51) Int. Cl. A61K 31/05; A61P 3/04; A61P 25/00

(52) U.S. Cl. 514/729

(57) ABSTRACT

The present invention relates to the use of one or more cannabinoids in the manufacture of medicaments for use in 0 the treatment of diseases and conditions benefiting from inverse agonism of the CB₁ and/or the CB₂ cannabinoid receptor. Preferably the cannabinoid is a cannabidiol (CBD) type compound or derivative thereof.

The present invention relates to the use of one or more cannabinoids in the manufacture of medicaments for use in the treatment of diseases and conditions benefiting from inverse agonism of the CB₁ and/or the CB₂ cannabinoid receptor. Preferably the cannabinoid is a cannabidiol (CBD) type compound or derivative thereof. Preferably the diseases or conditions to be treated are taken from the group: obesity, schizophrenia, epilepsy, cognitive disorders such as Alzheimer's disease, bone disorders such as osteoporosis, bulimia, obesity associated with type II diabetes (non-insulin dependant diabetes), the treatment of drug, alcohol and nicotine abuse or dependency and inflammatory disorders. It also relates to a method of treating diseases and conditions benefiting from inverse agonism of the CB₁ and/or the CB₂ cannabinoid receptor. The invention further relates to a method of generating a cosmetically beneficial weight loss by inducing suppression of appetite. The invention still further relates to a food or drink supplement comprising an effective amount of one or more cannabinoids that cause inverse agonism of the CB₁ and/or the CB₂ cannabinoid receptor.

### BACKGROUND TO THE INVENTION

The action of many known cannabinoids can be attributed to their interaction with cannabinoid receptors. Cannabinoid receptors are present in mammalian systems and several classes of G-Protein coupled receptors have been identified. The receptors that are present mainly in the central nervous system are known as CB₁ receptors, whereas a different type of receptor, which are found substantially in the immune system, are known as the CB₂ receptors.

Cannabinoids are generally known to be cannabinoid receptor agonists. When a cannabinoid receptor agonist binds to a cannabinoid receptor a response is triggered. This response is known as a signalling pathway.

Compounds which are known to bind to the CB₁ cannabinoid receptor include delta-9-tetrahydrocannabinol (THC), R-(+)-WIN55212 and anandamide. These compounds are as such described as CB₁ agonists as when they bind to the CB₁ receptor a specific response is produced.

Agonism at a receptor will often lead to an active response by the cell. Many disease states result from the overactive or overabundant effects of agonists at their receptors.

Cannabinoid receptors are known to be constitutively active. This means that the receptors undergo some degree of coupling to their signalling pathways even in the absence of an agonist. As such they exhibit a background tone.

In the presence of an agonist this background tone is increased this can cause an intensification of a disease state that has resulted from the active response of the cell.

Research into compounds that are able to oppose the ability of such agonists has led to the discovery of compounds that act as cannabinoid receptor antagonists.

A neutral antagonist is a compound that will bind to the receptor but will lack any efficacy as a receptor agonist. Such a neutral antagonist will compete with agonists for its receptor and once bound will not result in any active response. In constitutively active receptors the background tone remains unaffected.

An inverse agonist will also bind to its receptor and will lack any efficacy as a receptor agonist. Once an inverse agonist is bound to a receptor it is able to produce an opposite effect of the active response. Therefore in constitutively active receptors an inverse agonist is able to either partially or completely switch off the background tone.

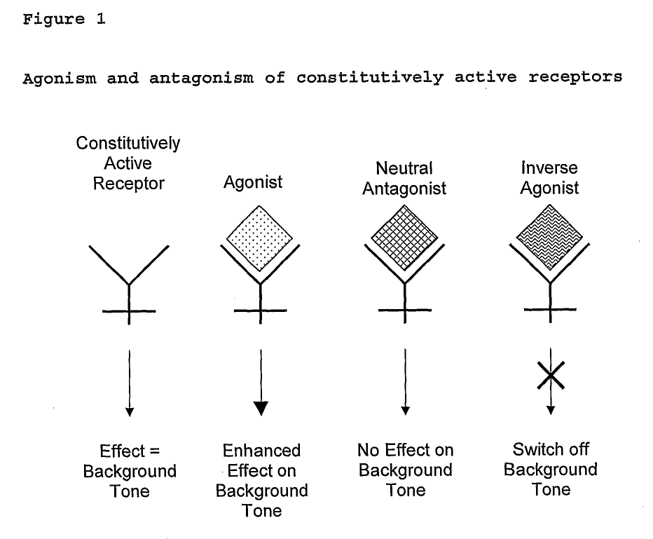

The way in which constitutively active receptors work in the presence of agonists and different types of receptor antagonists is shown in FIG. 1.

The ability of a compound to have inverse agonism properties of a constitutively active receptor may be extremely beneficial in the treatment of diseases where a change in the background tone of a cell is the cause of the disease state.

Examples of diseases and conditions that are the result of the background tone of constitutively active cannabinoid receptors include but are not limited to obesity, schizophrenia, epilepsy, cognitive disorders such as Alzheimer's disease, bone disorders such as osteoporosis, bulimia, obesity associated with type II diabetes (non-insulin dependant diabetes), the treatment of drug, alcohol and nicotine abuse or dependency and inflammatory disorders (Pertwee, R. G., 2000).

There is evidence that the endogenous CB₁ agonist, anandamide, is released in the brain to mediate processes such as feeding and appetite (Di Marzo et al., 2001). This raises the possibility that a CB₁ receptor inverse agonist could be effective in the clinic as an appetite suppressant.

One such cannabinoid receptor inverse agonist is SR141716A. The use of this compound in the regulation of appetite has been described by Maruani and Soubrie in U.S. Pat. No. 6,444,474 and EP0969835.

The compound SR141716A is a synthetic compound and as such its long-term effects cannot be completely quantified by clinical trials. It is not known how a synthetic compound such as this will interfere with the cannabinoid receptors on a very long-term basis (it is likely from data accumulated in a clinical study with SR141716A that appetite suppressant treatments will have to be chronic). The clinical study showed a significant increase in depression in at least some of the patients enrolled in the trials. Also a recent article in the journal Multiple Sclerosis describes a patient whose previously subclinical case of multiple sclerosis became active when treatment with SR141716A was started.

Naturally occurring CB₁ and CB₂ receptor inverse agonists which are produced by the cannabis plant are likely to have a less complex pharmacology than those of an inverse agonist which has been chemically synthesised to bind with the cannabinoid receptor. This is because the human body has been in contact with such substances for millennia and as such the bodies pharmacological systems have developed in the presence of plant cannabinoids and if there were any untoward side effects these would be known already. However, until the present time none of the cannabinoids produced by the cannabis plant have been found to possess inverse agonism properties of the cannabinoid receptor.

Surprisingly the applicants have shown that the cannabinoid cannabidiol (CBD) is an inverse agonist of both the CB₁ and CB₂ cannabinoid receptors.

The cannabinoid CBD is a phytocannabinoid, which unlike THC does not produce psychoactive effects in users.

The finding that CBD acts as an inverse agonist of both the CB₁ and CB₂ receptors was particularly surprising, as it has previously been shown to have low affinities for both cannabinoid receptors. This suggests that the inverse agonism of the receptors may take place at a site away from the binding site on the cannabinoid receptor.

The applicants have also found that in preclinical studies CBD is able to produce an appetite suppressant effect in mammals.

### SUMMARY OF THE INVENTION

According to the first aspect of the present invention there is provided the use of one or more cannabinoids in the manufacture of a medicament for use in the treatment or prevention of a disease or a condition benefiting from inverse agonism of the CB₁ and/or the CB₂ cannabinoid receptor.

Preferably the one or more cannabinoids is a cannabidiol (CBD) type compound or a derivative thereof.

References to CBD, CBD type compounds or derivatives thereof, particularly with regard to therapeutic use, will be understood to also encompass pharmaceutically acceptable salts of such compounds. The term “pharmaceutically acceptable salts” refers to salts or esters prepared from pharmaceutically acceptable non-toxic bases or acids, including inorganic bases or acids and organic bases or acids, as would be well known to persons skilled in the art. Many suitable inorganic and organic bases are known in the art.

Cannabinoid biosynthesis begins when a precursor molecule reacts with geranylpyrophosphate to form a ringed structure. As shown below, CBD type compounds are mostly 21 carbon compounds.

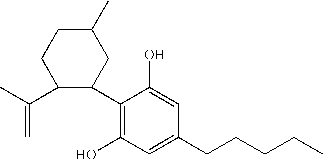

Variation in the length of the side chain that is attached to the aromatic ring (bottom right hand side of the structure) can produce different types of CBD compounds. For example when the side chain is a pentyl (5 carbon) chain the compound produced will be CBD. If the pentyl chain is replaced with a propyl (3 carbon) chain the CBD type compound formed is CBDV (cannabidivarin). The propyl variant will be formed if a 10 carbon precursor is reacted at the first stage of the biosynthetic pathway rather than a 12 carbon compound.

Synthetic variants of CBD include dimethylheptyl CBD. This compound also has variations in the side chain of the CBD skeleton.

The scope of the invention also extends to derivatives of CBD that retain the desired activity of inverse agonism of the CB₁ and or CB₂ receptor. Derivatives that retain substantially the same activity as the starting material, or more preferably exhibit improved activity, may be produced according to standard principles of medicinal chemistry, which are well known in the art. Such derivatives may exhibit a lesser degree of activity than the starting material, so long as they retain sufficient activity to be therapeutically effective. Derivatives may exhibit improvements in other properties that are desirable in pharmaceutical active agents such as, for example, improved solubility, reduced toxicity, enhanced uptake, etc.

Preferably the disease or condition to be treated are taken from the group comprising: obesity, schizophrenia, epilepsy, cognitive disorders such as Alzheimer's disease, bone disorders such as osteoporosis, bulimia, obesity associated with type II diabetes (non-insulin dependant diabetes), the treatment of drug, alcohol and nicotine abuse or dependency and inflammatory disorders.

More preferably the inverse agonism of the CB₁ and/or the CB₂ cannabinoid receptor acts to suppress appetite.

Preferably the inverse agonism of the CB₁ and or CB₂ occurs at a site distinct from the CB₁ and or CB₂ cannabinoid receptor.

Preferably the one or more cannabinoids is in the form of an extract prepared from at least one cannabis plant.

More preferably the extract from at least one cannabis plant is a botanical drug substance.

Preferably the extract from at least one cannabis plant is produced by extraction with supercritical or subcritical CO₂.

Alternatively the extract from at least one cannabis plant is produced by contacting plant material with a heated gas at a temperature which is greater than 100° C., sufficient to volatilise one or more of the cannabinoids in the plant material to form a vapour, and condensing the vapour to form an extract.

Preferably the extract from at least one cannabis plant comprises all of the naturally occurring cannabinoids in the plant.

Alternatively the one or more cannabinoids are in a substantially pure or isolated form.

A “substantially pure” preparation of cannabinoid is defined as a preparation having a chromatographic purity (of the desired cannabinoid) of greater than 90%, more preferably greater than 95%, more preferably greater than 96%, more preferably greater than 97%, more preferably greater than 98%, more preferably greater than 99% and most preferably greater than 99.5%, as determined by area normalisation of an HPLC profile.

Preferably the substantially pure one or more cannabinoids used in the invention are substantially free of any other naturally occurring or synthetic cannabinoids, including cannabinoids that occur naturally in cannabis plants. In this context “substantially free” can be taken to mean that no cannabinoids other than the active one or more cannabinoids are detectable by HPLC.

In another aspect of the present invention the one or more cannabinoids are in a synthetic form.

Preferably the one or more cannabinoids are formulated as a pharmaceutical composition further comprising one or more pharmaceutically acceptable carriers, excipients or diluents.

Alternatively the one or more cannabinoids are formulated as a food or drink supplement further comprising one or more acceptable carriers, excipients or diluents.

The invention also encompasses pharmaceutical compositions comprising CBD type compound or derivative thereof, or pharmaceutically acceptable salts or derivatives thereof, formulated into pharmaceutical dosage forms, together with suitable pharmaceutically acceptable carriers, such as diluents, fillers, salts, buffers, stabilizers, solubilisers, etc. The dosage form may contain other pharmaceutically acceptable excipients for modifying conditions such as pH, osmolarity, taste, viscosity, sterility, lipophilicity, solubility etc. The choice of diluents, carriers or excipients will depend on the desired dosage form, which may in turn be dependent on the intended route of administration to a patient.

Suitable dosage forms include, but are not limited to, solid dosage forms, for example tablets, capsules, powders, dispersible granules, cachets and suppositories, including sustained release and delayed release formulations. Powders and tablets will generally comprise from about 5% to about 70% active ingredient. Suitable solid carriers and excipients are generally known in the art and include, e.g. magnesium carbonate, magnesium stearate, talc, sugar, lactose, etc. Tablets, powders, cachets and capsules are all suitable dosage forms for oral administration.

Liquid dosage forms include solutions, suspensions and emulsions. Liquid form preparations may be administered by intravenous, intracerebral, intraperitoneal, parenteral or intramuscular injection or infusion. Sterile injectable formulations may comprise a sterile solution or suspension of the active agent in a non-toxic, pharmaceutically acceptable diluent or solvent. Liquid dosage forms also include solutions or sprays for intranasal, buccal or sublingual administration. Aerosol preparations suitable for inhalation may include solutions and solids in powder form, which may be combined with a pharmaceutically acceptable carrier, such as an inert compressed gas.

Also encompassed are dosage forms for transdermal administration, including creams, lotions, aerosols and/or emulsions. These dosage forms may be included in transdermal patches of the matrix or reservoir type, which are generally known in the art.

Pharmaceutical preparations may be conveniently prepared in unit dosage form, according to standard procedures of pharmaceutical formulation. The quantity of active compound per unit dose may be varied according to the nature of the active compound and the intended dosage regime. Generally this will be within the range of from 0.1 mg to 1000 mg.

According to a second aspect of the present invention there is provided a method for the treatment or prevention of diseases benefiting from inverse agonism of the CB₁ and/or CB₂ receptor, which comprises administering to a subject in need thereof an effective amount of one or more cannabinoids.

In another embodiment, the invention relates to a method of administering a CBD type compound or derivative thereof to a patient for the treatment or prevention of a disease caused by constitutive activity of the CB₁ and/or the CB₂ cannabinoid receptor, such as for example obesity, schizophrenia, epilepsy or cognitive disorders such as Alzheimer's disease, bone disorders, bulimia, obesity associated with type II diabetes (non-insulin dependant diabetes), drug, alcohol or nicotine abuse or dependency or inflammatory disorders.

According to a third aspect of the present invention there is provided a method for generating a cosmetically beneficial weight loss by inducing suppression of appetite in a subject comprising administering to the subject an effective amount of one or more cannabinoids which cause inverse agonism of the CB₁ and/or CB₂ receptor.

Preferably the one or more cannabinoids is a CBD type compound or a derivative thereof.

The invention still further extends to use of a CBD type compound or derivative thereof as an appetite suppressant. In certain circumstances the appetite suppressant may be utilised in order to achieve a cosmetically beneficial loss of weight in a human subject, without necessarily producing medical or therapeutic benefit to that subject. In this context administration of the appetite suppressant may not be construed as a medical or therapeutic treatment of the subject.

According to a fourth aspect of the present invention there is provided a food or drink supplement comprising an effective amount of one or more cannabinoids which cause inverse agonism of the CB₁ and/or CB₂ receptor.

Preferably the one or more cannabinoids is a CBD type compound or a derivative thereof.

### SPECIFIC DESCRIPTION

The examples detailed below describe studies undertaken to investigate the properties of CBD at the CB₁ and CB₂ cannabinoid receptors. In particular the ability of CBD to alter the background tone of the constitutively active CB₁ and CB₂ receptors was investigated. Furthermore the therapeutic usefulness of CBD with reference to the treatment of diseases or conditions benefiting from inverse agonism of the CB₁ and CB₂ cannabinoid receptors was undertaken.

Certain aspects of this invention are further described, by way of example only, with reference to the accompanying drawings in which:

FIG. 1 shows the agonism and antagonism of constitutively active receptors.

### EXAMPLE 1

### Investigation into the Properties of CBD at the CB₁ and CB₂ Receptors

The major constituent of cannabis delta-9-tetrahydrocannabinol (THC) has been well investigated as a medicinal substance yet its therapeutic usefulness can often be hindered by its additional psychotropic activity. This often limits the amount of THC that can be administered to a patient.

In contrast the naturally occurring plant cannabinoid cannabidiol (CBD) has been less well documented therapeutically, although it is known that CBD is non-psychoactive and has a low affinity for both the CB₁ and the CB₂ cannabinoid receptors.

Previously it has been shown that CBD is able to antagonize electrically evoked contractions of the mouse isolated vas deferens by a non-CB₁ mediated mechanism by acting pre-junctionally at non-CB₁ sites (Pertwee, 2002). This previous data using the mouse vas deferens suggested that CBD was competing with the known CB₁ receptor agonist WIN55212 for a non-CB₁ site.

As CBDs antagonism effects of WIN55212 occurred pre-junctionally and as CBD was known to have a low affinity for both the CB₁ and the CB₂ cannabinoid receptors it was not thought that this cannabinoid would possess properties that meant that it was able to produce inverse agonism at the constitutively active CB₁ and CB₂ receptors.

The current study investigated the effects of CBD at the CB₁ and CB₂ receptors themselves. CB₁ receptors in brain tissue and CB₂ receptors in CHO cell membranes transfected with human CB₂ receptors were used to compare the properties of CBD with the established CB₁ receptor inverse agonist SR141716A and the known CB₂ receptor inverse agonist SR144528.

### Method:

The test articles used were: CBD (purified plant extract), SR141716A (known CB₁ receptor inverse agonist) and SR144528 (known CB₂ receptor inverse agonist). The compounds were dissolved in DMSA prior to use.

Whole mouse brain membranes were prepared as described by Thomas et al., 2004. CHO cells were stably transfected with cDNA encoding human cannabinoid CB₂ receptors and were maintained at 37° C. and 5% CO₂ in Dulbecco's Modified Eagle's Medium nutrient mixture.

### Radioligand Displacement Assay

The assays were carried out with the established CB₁ and CB₂ cannabinoid receptor agonist CP55940. This was radiolabelled to form [³H]CP55940.

Binding of the radiolabelled compound was initiated by the addition of either the brain membranes (33 μg protein per tube) or the transfected hCB₂ cells (25 μg protein per tube).

All assays were performed at 37° C. for 60 min before termination by addition of ice-cold wash buffer (50 mM Tris buffer, 1 mg ml⁻¹ bovine serum albumin, pH 7.4) and vacuum filtration using a 24-well sampling manifold and GF/B filters that had been soaked in wash buffer at 4° C. for at least 24 h.

### [³⁵S]GTPγS Binding Assay

The assays were carried out with GTPγS binding buffer (50 mM Tris-HCl; 50 mM Tris-Base; 5 mM MgCl₂; 1 mM EDTA; 100 mM NaCl; 1 mM DTT; 0.1% BSA) in the presence of [³⁵S]GTPγS and GDP, in a final volume of 500 μl. Binding was initiated by the addition of [³⁵S]GTPγS to the tubes. The drugs were incubated in the assay for 60 min at 30° C. The reaction was terminated by a rapid vacuum filtration method using Tris buffer (50 mM Tris-HCl; 50 mM Tris-Base; 0.1% BSA), as described previously, and the radioactivity was quantified by liquid scintillation spectrometry.

The agonism of the CB₁ or CB₂ receptors by CP55940 results in a response in the cell. This response is the binding of [³⁵S]GTPγS to the cell membrane.

Changes in the response in the presence of the test compound can be measured in order to determine whether the compound is acting as an agonist, a neutral antagonist or an inverse agonist. As is described in FIG. 1 an agonist will increase the response, a neutral antagonist will have no effect on the response and an inverse agonist will stop or reverse the response. The K_{B}-value that results from these investigations is therefore an indicator of the cells response.

The test compounds were also tested to determine whether they were able to displace the agonist CP55940 from the binding site of the CB₁ or CB₂ receptor. The Kᵢ-value that resulted from this investigation gives an insight into how strongly the test compound competes with the agonist for the binding site.

### Results:

1. Experiments Conducted with Whole Mouse Membranes to Determine Activity at the CB₁ Receptor

Table 1 describes the data produced by CBD and the known CB₁ receptor inverse agonist SR141716A at the CB₁ receptor.

The table describes the K_{B}-values for the CP55940 induced activation of [³⁵S]GTPγS binding to the cell membrane in the presence of the known CB₁ receptor inverse agonist and CBD. The Kᵢ-value for the displacement of the [³H]CP55940 from the membranes is also shown.

TABLE 1

| | K_{B}-value for | Kᵢ-value for |
|---|---|---|
| Test Article | Binding | Displacement |
| SR141716A | 0.09 nM | 2.2 nM |
| (10 nM) | | |
| CBD | 78.8 nM | 4.9 μM |
| (1 μM) | | |

Both SR141617A and CBD were able to produce a rightward shift in the log-concentration response curve of the established CB₁/CB₂ receptor agonist CP55940 in the mouse brain membranes when the measured response was stimulation of [³⁵S]GTPγS binding. These data show that both compounds were able to inhibit the response caused by the activation of the CB₁ receptor by CP55940.

The K_{B}-value of SR141716A was 0.09 nM which is only slightly less than its CB₁ Kᵢ-value of 2.2 nM for the displacement of [³H]CP55940 from the mouse brain membranes. This infers that this compound is able to produce an inverse response in the cell at a similar concentration to that at which it competes and binds to the receptor.

However the K_{B}-value of CBD was 78.8 nM this was well below its CB₁ Kᵢ-value of 4.9 μM for the displacement of [³H]CP55940 from the mouse brain membranes. These data show that CBD is able to act as an inverse agonist at the CB₁ receptor. They also show that CBD is able to act as an inverse agonist at concentrations much below that at which it will compete with the agonist for the binding site.

This property may be of significant value as it infers that CBD will form a less strong interaction with the cannabinoid receptor in vivo and as such is likely to produce fewer side effects in use than the compound SR141716A.

Further experiments were undertaken at different concentrations of the test compounds. At concentrations of 1 and 10 μM CBD produced a significant inhibition of [³⁵S]GTPγS binding to the mouse brain membrane. The inhibitory effect of CBD at 1 μM was similar to that of SR141716A at 1 μM, whereas the inhibitory effect of CBD at 10 μM greatly exceeded that of SR141716A at the same concentration. At the higher concentration CBD is a more potent inverse agonist of the CB₁ receptor than SR141716A.

2. Experiments Conducted with CHO Cell Membranes to Determine Activity at the CB₂ Receptor

Table 2 describes the data produced by CBD and the known CB₂ receptor inverse agonist SR144528 at the CB₂ receptor.

The table describes the K_{B}-values for the CP55940 induced activation of [³⁵S]GTPγS binding to the cell membrane in the presence of the known CB₁ receptor inverse agonist and CBD. The Kᵢ-value for displacement of the [³H]CP55940 from the membranes is also shown.

TABLE 2

| | K_{B}-value for | Kᵢ-value for |
|---|---|---|
| Test Article | binding | displacement |
| SR144528 | 0.49 nM | 7.5 nM |
| (100 nM) | | |
| CBD | 65.1 nM | 4.2 μM |
| (1 μM) | | |

Both SR144528 and CBD were able to produce a downward and rightward shift in the log-concentration response curve of the established CB₁/CB₂ receptor agonist CP55940 in the CHO cell membranes when the measured response was stimulation of [³⁵S]GTPγS binding. These data show that both compounds were able to inhibit the response caused by the activation of the CB₂ receptor by CP55940.

The K_{B}-value of SR144528 was 0.49 nM which was 15 times less than its CB₁ Kᵢ-value of 7.5 nM for the displacement of [³H]CP55940 from the CHO cell membranes.

The K_{B}-value of CBD was 65.1 nM which was 65 times less than its CB₁ Kᵢ-value of 4.2 μM for the displacement of [³H]CP55940 from the CHO cell membranes.

These data show that CBD is also able to act as an inverse agonist at the CB₂ receptor. They also show that both CBD and SR144528 are both able to act as inverse agonists at concentrations below that at which they compete with the agonist for the binding site. However CBD was shown to compete at a far lower concentration than SR144528.

In summary the data produced in this example indicates that CBD is an inverse agonist at both the CB1 and CB2 receptors. It is also shown that CBD will only displace agonists from their cannabinoid receptor binding sites at far higher concentrations than that at which it is able to produce the inverse agonism in the cell. Because of this CBD is a much better candidate for use in the clinic as it is less likely to disturb the endogenous cannabinoid system that is operational in all mammals.

### EXAMPLE 2

### Investigation into the Ability of Cannabidiol to Influence Appetency in Rats

The data described in Example 1 above demonstrate that CBD is a potent inverse agonist of the CB₁ cannabinoid receptor. Inverse agonism of the CB₁ receptor is thought to be helpful in the regulation of appetite as the known CB₁ receptor inverse agonist SR141716A has been found in clinical studies to be helpful in reducing bodyweight in obese subjects.

The inverse agonism of the constitutively active CB₁ and CB₂ cannabinoid receptors is also thought to be useful in the treatment or prevention of other diseases or conditions as it is known that over-production of endogenous cannabinoids are implicated in such diseases. Such diseases include but are not limited to: obesity, schizophrenia, epilepsy, cognitive disorders such as Alzheimer's disease, bone disorders such as osteoporosis, bulimia, obesity associated with type II diabetes (non-insulin dependant diabetes), the treatment of drug, alcohol and nicotine abuse or dependency and inflammatory disorders.

The inverse agonism of either the CB₁ or CB₂ receptor or both receptors in such diseases means that the response in the cell caused by the endocannabinoids is either halted or reversed.

As is shown in Example 1 above, CBD is able to act as an inverse agonist at the CB₁ and CB₂ receptors. In order to evaluate this property against the diseases and conditions that are known to benefit from such inverse agonism of the cannabinoid receptors it was decided that a preclinical study whereby the bodyweight gain, food consumption and food conversion efficiency were measured was undertaken.

The study was performed in male and female rats over a 104 week study.

### Method:

The test article used was CBD (whole plant extract), this was administered to the rats orally in their diet during the 104 weeks of the study. Dose levels used were 5, 15 and 50 mg/kg/day of CBD. Control animals did not receive any test article in their diet.

The CBD whole plant extract comprises along with the major cannabinoid CBD, other cannabinoids and non-cannabinoid components. These are listed in the table below:

CBD-containing plant extract
(% w/w of extract)

Major/Minor Cannabinoid:

| | |
|---|---|
| THC Content | 2.0-6.5 |
| CBD Content | 57.0-72.0 |

Other Cannabinoids:

| | |
|---|---|
| Cannabigerol | 0.8-6.5 |
| Cannabichromene | 3.0-6.5 |
| Tetrahyrocannabidivarin | — |
| Tetrahydrocannabinolic acid | — |
| Cannabidivarin | 1.0-2.0 |
| Cannabidiolic acid | <2.0 |

Terpenes:

| | |
|---|---|
| Monoterpenes | 0.4 |
| Di/tri-terpenes | 0.4 |
| Sesquiterpenes | 2.0 |
| Other terpenes | <3.0 |

Other minor plant derived components including:

| | | |
|---|---|---|
| Sterols | | |
| Triglycerides | | |
| Alkanes | | |
| Squalene | {close oversize brace} | 1.7-28.4 |
| Tocopherol | | |
| Carotenoids | | |

The HsdBrlHan:WIST derived strain of rats were randomly allocated to a study group comprising 50 animals of each sex per group.

Bodyweights and food consumption were measured weekly for the first 16 weeks of the study and then every four weeks thereafter.

### Results:

### 1. Bodyweight Gain

Table 3 below details the mean bodyweight gain in grams over the study period and as a percentage of the control animals.

TABLE 3

| | Bodyweight Gain (g) [Percentage of control] | | | | | | | | | | | | | |
|---|---|---|---|---|---|---|---|---|---|---|---|---|---|---|
| Period | Dose (g) [sex] | | | | | | | | | | | | | |
| (weeks) | 0 [M] | 5 [M] | | 15 [M] | | 50 [M] | | 0 [F] | 5 [F] | | 15 [F] | | 50 [F] | |
| 1-13 | 218.9 | 215.2 | [98] | 200.8 | [92] | 181.7 | [83] | 91.1 | 90.7 | [100] | 87.5 | [96] | 79.4 | [87] |
| 1-27 | 284.4 | 281.6 | [99] | 260.4 | [92] | 232.6 | [82] | 118.7 | 114.2 | [96] | 111.9 | [94] | 98.0 | [83] |
| 1-51 | 355.9 | 352.3 | [99] | 324.6 | [91] | 287.9 | [81] | 166.1 | 164.5 | [99] | 147.0 | [89] | 125.7 | [76] |
| 51-104 | 94.0 | 91.5 | [97] | 66.8 | [71] | 46.3 | [49] | 93.5 | 78.1 | [84] | 67.3 | [72] | 42.8 | [46] |
| 1-104 | 449.9 | 443.8 | [99] | 391.4 | [87] | 334.2 | [74] | 259.6 | 242.6 | [93] | 2143 | [83] | 168.5 | [65] |

As can be observed in Table 3 there was a dosage-related reduction in overall bodyweight gain between the start of the study at week 1 and the end of the study at week 104. This reduction in bodyweight gain was observed in males at the 15 and 50 mg/kg/day doses and for females at all dose levels.

### 2. Food Consumption

Table 4 below details the mean amount of food that was consumed in grams by each group over the study period.

TABLE 4

| | Food consumption (g/animal/day) [Percentage of control] | | | | | | | | | | | | | |
|---|---|---|---|---|---|---|---|---|---|---|---|---|---|---|
| Period | Dose (g) [sex] | | | | | | | | | | | | | |
| (weeks) | 0 [M] | 5 [M] | | 15 [M] | | 50 [M] | | 0 [F] | 5 [F] | | 15 [F] | | 50 [F] | |
| 1-16 | 20.7 | 20.2 | [98] | 19.3 | [93] | 18.4 | [89] | 16.6 | 16.1 | [97] | 15.9 | [96] | 14.7 | [89] |
| 1-104 | 20.5 | 20.2 | [98] | 19.3 | [94] | 18.4 | [90] | 17.0 | 16.7 | [98] | 16.1 | [95] | 15.0 | [88] |

It was observed that the amount of food consumed by both the male and the female animals administered 15 and 50 mg/kg/day decreased in comparison to the control.

### 3. Food Conversion Efficiency

Table 5 below details the mean food conversion efficiency of the study animals over the study period. The food conversion efficiency value is calculated to determine the weight of food in grams required to increase the bodyweight of the animal by one gram.

TABLE 5

| | Food conversion efficiency (g) [Percentage of control] | | | | | | | |
|---|---|---|---|---|---|---|---|---|
| Period | Dose (g) [sex] | | | | | | | |
| (weeks) | 0 [M] | 5 [M] | 15 [M] | 50 [M] | 0 [F] | 5 [F] | 15 [F] | 50 [F] |
| 1-104 | 3.01 | 3.02 [100] | 2.79 [93] | 2.49 [83] | 2.10 | 2.00 [95] | 1.83 [87] | 1.54 [73] |

As can be seen from Table 5 there is a clear decrease in the food conversion efficiency value over the study period. The decrease was dosage related for females given 15 and 50 mg/kg/day of CBD and for males at 50 mg/kg/day.

In conclusion the administration of CBD whole plant extract to the study animals at both 15 and 50 mg/kg/day had a marked effect on overall bodyweight gain. Both groups, equally for males and females, showed a greater than 10% decrease in bodyweight over the study period in comparison to the controls. The striking reduction in food conversion efficiency in these dose groups infers that the reduction in bodyweight was not purely due to inappetance of the diet.

To conclude the data presented in the Examples above show that CBD is a potent inverse agonist at both the CB₁ and CB₂ cannabinoid receptors. These data demonstrate this property both in vitro and in vivo. As such this naturally occurring cannabinoid has real potential for use in the treatment or prevention of diseases benefiting from inverse agonism of the CB₁ and or the CB₂ cannabinoid receptor.

1.-10. (canceled)

11. A method for the treatment or prevention of a disease or condition benefiting from inverse agonism of the CB₁ and/or CB₂ cannabinoid receptor, which comprises administering to a subject in need thereof a therapeutically effective amount of cannabidiol (CBD) type compound or a derivative thereof, wherein the disease or condition benefiting from inverse agonism of the CB₁ and/or the CB₂ cannabinoid receptor is selected from the group consisting of: obesity, cognitive disorders such as Alzheimer's disease, bulimia, obesity associated with type II diabetes (non-insulin dependant diabetes), and the treatment of drug, alcohol or nicotine abuse or dependency.

12. (canceled)

13. A method of generating a cosmetically beneficial weight loss by inducing suppression of appetite in a subject comprising administering to the subject an effective amount of one or more cannabinoids which cause inverse agonism of the CB₁ and/or CB₂ cannabinoid receptor, wherein the one or more cannabinoids is a cannabidiol (CBD) type compound or a derivative thereof.

14. A food or drink supplement comprising an effective amount of one or more cannabinoids which cause inverse agonism of the CB₁ and/or CB₂ cannabinoid receptor.

15. A food or drink supplement as claimed in claim 14, wherein the one or more cannabinoids is a cannabidiol (CBD) type compound or a derivative thereof.

16. The method as claimed in claim 11, wherein the inverse agonism of the CB₁ and/or CB₂ cannabinoid receptor occurs at a site distinct from the CB₁ and/or CB₂ cannabinoid receptor.

17. The method as claimed claim 11, wherein the cannabidiol (CBD) type compound or a derivative thereof are in the form of an extract prepared from at least one cannabis plant.

18. The method as claimed in claim 17, wherein the extract prepared from at least one cannabis plant is in the form of a botanical drug substance.

19. The method as claimed in claim 17, wherein the extract prepared from at least one cannabis plant comprises all of the naturally occurring cannabinoids in said at least one cannabis plant.

20. The method as claimed in claim 11, wherein the cannabidiol (CBD) type compound or a derivative thereof are in a substantially pure or isolated form.

21. The method as claimed in claim 11, wherein the cannabidiol (CBD) type compound or a derivative thereof are in a synthetic form.

22. The method as claimed in claim 11, wherein the cannabidiol (CBD) type compound or a derivative thereof are formulated as a pharmaceutical composition further comprising one or more pharmaceutically acceptable carriers, excipients or diluents.

23. The method as claimed in claim 11, wherein the cannabidiol (CBD) type compound or a derivative thereof are formulated as a food or drink supplement further comprising one or more acceptable carriers, excipients or diluents.

24. The method as claimed in claim 13, wherein the inverse agonism of the CB₁ and/or CB₂ cannabinoid receptor occurs at a site distinct from the CB₁ and/or CB₂ cannabinoid receptor.

25. The method as claimed claim 13, wherein the cannabidiol (CBD) type compound or a derivative thereof are in the form of an extract prepared from at least one cannabis plant.

26. The method as claimed in claim 25, wherein the extract prepared from at least one cannabis plant is in the form of a botanical drug substance.

27. The method as claimed in claim 25, wherein the extract prepared from at least one cannabis plant comprises all of the naturally occurring cannabinoids in said at least one cannabis plant.

28. The method as claimed in claim 13, wherein the cannabidiol (CBD) type compound or a derivative thereof are in a substantially pure or isolated form.

29. The method as claimed in claim 13, wherein the cannabidiol (CBD) type compound or a derivative thereof are in a synthetic form.

30. The method as claimed claim 13, wherein the cannabidiol (CBD) type compound or a derivative thereof are formulated as a pharmaceutical composition further comprising one or more pharmaceutically acceptable carriers, excipients or diluents.

31. The method as claimed in claim 13, wherein the cannabidiol (CBD) type compound or a derivative thereof are formulated as a food or drink supplement further comprising one or more acceptable carriers, excipients or diluents.
